(19) United States
(12) Patent Application Publication
(10) Pub. No.: US 2026/0334974 A1
(43) Pub. Date: Oct. 29, 2026

(54) SCREW IMPLANT SYSTEM

(71) Applicant: Diabetic Orthopaedics, LLC, Monmouth Beach, NY (US)

(72) Inventors: Christopher GROSS, Charleston, SC (US); Jakob SCHLOSSER, Whitehall, PA (US); Mason B. GUNKLE, Northampton, PA (US); Dina GOFMAN, Fair Lawn, NJ (US); Darren HUFFMAN, Newtown, CT (US); Matthew Christopher CUGLIARI, Nutley, NJ (US); Jose Alexander RODRIGUEZ, Elizabeth, NJ (US); Edward Arthur GARDINIER, Bethlehem, PA (US); Christie Stefany Ortega, Newark, NY (US)

(73) Assignee: Diabetic Orthopaedics, LLC, Monmouth Beach, NJ (US)

(21) Appl. No.: 19/193,794

(22) Filed: Apr. 29, 2025

(51) Int. Cl. A61B 17/34; A61B 17/00

(52) U.S. Cl. CPC A61B 17/3472; A61B 2017/00893

(57) ABSTRACT

There is disclosed an implant comprising a body section having a hollow internal region, a plunger configured to be inserted inside of said body section. The plunger is substantially cylindrical and has first section having a first diameter and a second section having a second diameter wherein said first diameter is smaller than the second diameter. When the plunger is inserted into the body section it inserts into said internal hollow region of the body section to serve as a strengthening rod for said body section. In at least one embodiment, there can be a biological solution that is inserted into the implant. In another embodiment there can be a spring to bias the plunger inside of the body section.

### BACKGROUND OF THE INVENTION

For years, implants have been placed into a person's body without any system for advanced healing or interaction with adjacent bony matter. When these implants are placed into a person's body, they can at times become dislodged, fracture, and/or result in improper healing based upon excessive loading. Therefore, there is a need for an implant that is configured to aid in the healing of the surgical area as well as to improve bone on bone/implant interaction. Under certain circumstances, these implants are implanted into regions that do not receive a significant amount of blood flow or into regions with poor bone quality. Therefore, these implants may require additional aids such as biological material or dynamic compression by the implant to force an additional bone on implant/bone interaction.

### SUMMARY

One embodiment discloses a screw-type implant that is configured to fixate at least one bony region to another via connecting threads on each side. In at least one embodiment, there is a solution delivery system which is configured to be delivered when the implant is set in place inside of a patient. The solution delivery system is configured to deliver bone growth factors or biologic solution to the region that is receiving an operation to promote bone growth. Additionally, once the biologic solution is delivered, the cannulated implant becomes a solid implant, allowing for increased strength and rigidity.

In another embodiment, there is a screw-type implant that relies on a biasing internal element such as a spring to improve the connection between two adjacent bones for increased bone apposition during the bone healing and remodeling process.

### BRIEF DESCRIPTION OF THE DRAWINGS

Other objects and features of the present invention will become apparent from the following detailed description considered in connection with the accompanying drawings, which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1A is a side perspective exploded view of a first embodiment;

FIG. 1B is another side perspective view which shows the holes in a spiral pattern;

FIG. 1C is a perspective view of another embodiment;

FIG. 1D is a side exploded view of another embodiment;

FIG. 2A is a side view of the first embodiment of FIG. 1 with the two parts put at least partially assembled;

FIG. 2B is a side cross-sectional view of the embodiment of FIG. 1 with the two parts put at least partially assembled;

FIG. 2C is a side cross-sectional view of the body section which shows the body section without the plunger inserted;

FIG. 2D is an end view of the biological solution storage well;

FIG. 3A is a side view of the embodiment of FIG. 1 which has the two parts fully assembled;

FIG. 3B is a side cross-sectional view of the embodiment of FIG. 1 which has the two parts fully assembled; the cannulated assembly is now a solid construct.

FIG. 3C is a side view of the embodiment of FIG. 1 with the addition of an end cap and displays the surgical placement;

FIG. 4A is a side perspective view of another embodiment of the second invention;

FIG. 4B is a side perspective view of the embodiment of FIG. 4A but having additional holes for releasing biological materials;

FIG. 5A is a side view of the parts of FIG. 4A, showing the device in its expanded state;

FIG. 5B is a side cross-sectional view of the two parts of FIG. 4A, showing the device in its expanded state;

FIG. 5C is a cross-sectional view of the profile of the internal face 139 of the body section 110;

FIG. 6A is a side view of the parts of FIG. 4, showing the device in its compressed state; and

FIG. 6B is a side cross-sectional view of the parts of FIG. 4 with the addition of an end cap, showing the device in its compressed state;

FIG. 6C is a side view of the embodiment of FIG. 4A with the addition of an end cap and displays the surgical placement;

FIG. 7A is a side view of the embodiment of FIG. 4A in an expanded state as shown in FIG. 5A;

FIG. 7B is a side view of the embodiment of FIG. 4A in a contracted state as shown in FIG. 6B;

FIG. 8 is a flow chart for a process for implementing the embodiment of FIG. 1A-1C;

FIG. 9 is a flow chart for a process for implementing the embodiment of FIG. 1D;

FIG. 10 is a flow chart for the process for implementing the embodiment of FIG. 4A.

### DETAILED DESCRIPTION

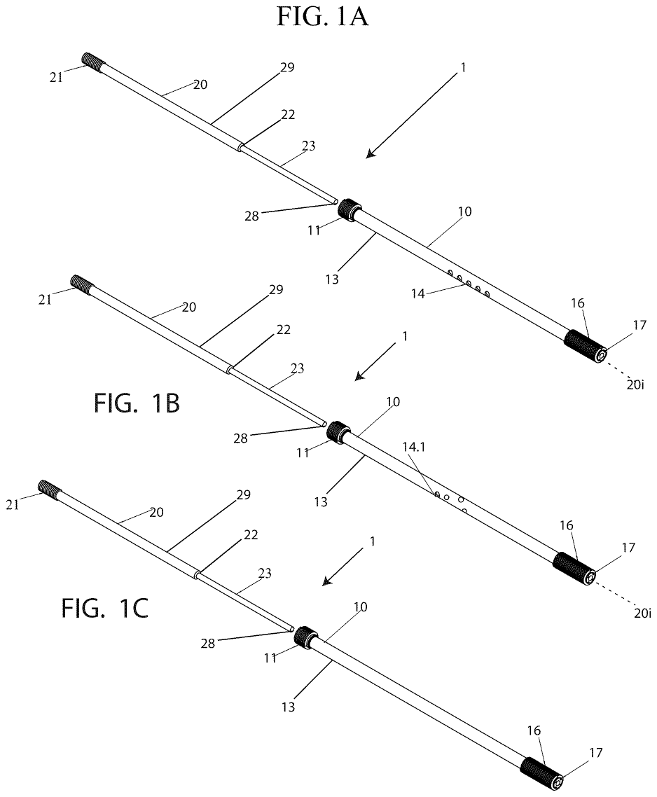

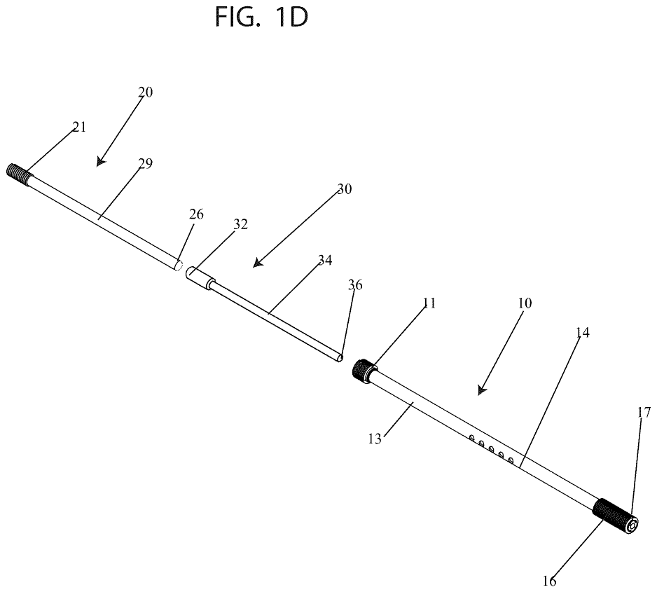

Referring in detail to the drawings, FIG. 1 is a side perspective exploded view of a first embodiment 1, including a first beam 10 which is an outer cylindrical shell that is configured to receive an inner strengthening rod or plunger 20. Outer shell or beam 10 has a first series of threads, or proximal threads 11, and an opposite or distal set of threads 16. Threads 16 are variable pitched relative to threads 11 in order to improve compression with the device. There is a cutting tip 17 positioned adjacent to the distal set of threads 16. In addition, spaced along the body section 13 of the beam 10 are a plurality of holes 14 to create openings from which a biological material will be released. The biological material can be in the form of stem cells, growth factors, or any other suitable biological material which is configured to aid in the healing of a patient's surgically affected area. The plunger 20 is configured to have a pre-assembled two-part but solid body comprising a first part 29 and a second part 23. Part 29 has a larger diameter compared to plunger section 23, and is located in the proximal section of the beaming system. When plunger 20 is inserted into beam 10, part 28 will be the first portion of plunger 20 to enter beam 10, and surface 22 will come into contact with section 15. As shown in FIGS. 1A and 1B the body section 13 extends along longitudinal axis 20i.

FIG. 1C is a side exploded view of another embodiment similar to that of FIG. 1A, however with this embodiment the body section 13 does not have holes 14. In addition, in this embodiment there is no biological solution housed inside of the body section 13.

FIG. 1D is a side perspective exploded view of first embodiment 1, including a two-part plunger system of plunger 20 and 30. Beam 10 is an outer cylindrical shell that is configured to receive an inner strengthening rod or plunger 30 prior to insertion of biological solution. The biological material can be in the form of stem cells, growth factors, or any other suitable biological material which is configured to aid in the healing of a patient's surgically affected area. Plunger 30 serves as a stopper to prevent biological solution from being released at the distal end, near end or tip 17. When the biological solution is inserted, plunger 20 is inserted to drive the biological solution that has been injected into this body section 13 out of holes 14.

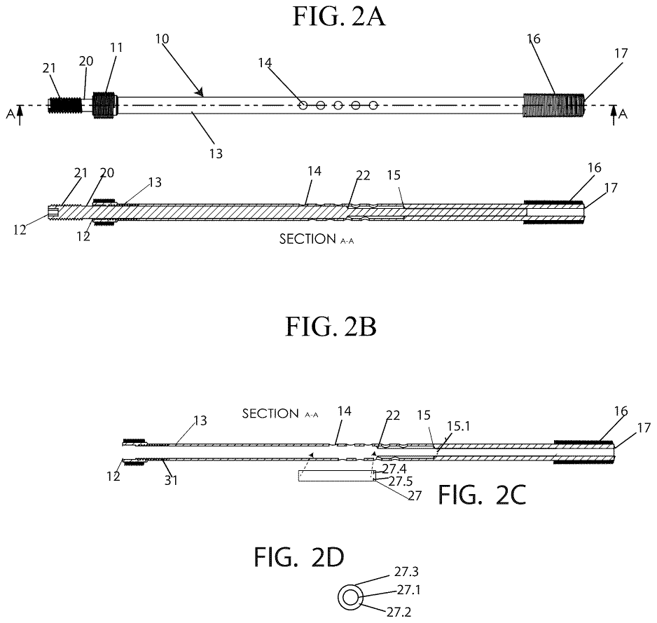

FIG. 2A is a side view of the first embodiment of FIG. 1 with the two parts partially assembled. In this view, plunger 20 is inserted into first beam 10 with threads 21. The outer body section 13 of first beam 10 has a plurality of holes 14 disposed therein. In this view, the plurality of holes 14 can be positioned in a plurality of different positions around the body section 13. For example, as shown in FIG. 2B the plurality of holes 14 can be positioned on opposite sides of the body section 13. Alternatively, as shown in FIG. 1B the holes 14.1 can be positioned in a spiral manner around the body section 13. As shown by the cross-sectional view taken along line A-A in FIG. 2B, stopper 15 may be added and positioned inside of body section 13. Section 15 has a smaller diameter compared to that of section 23. This narrowed opening forms a countersunk surface in which it is configured to receive a corresponding surface 22 on plunger 20. When plunger 20 fits inside of body section 13 it can be used to drive the biological solution that has been injected into this body section 13 out of holes 14. The biological solution can be either injected into body section 13 after the body section 13 has been inserted into the proximal bone and then into the distal bone, or the body section 13 can contain the biological solution therein either in a reservoir or have it stationed in side of the body section 13 via a gel. The holes 14 are sized such that they are larger than the diameter of the plunger section 23 thereby facilitating the flow of biological solution outside of the body section once the plunger 20 is placed inside.

If the biological solution is disposed inside of the body section 13 in a container, the second part or plunger section 23 can then inserted into body section 13 and then be used to puncture or release a container of biological solution. This solution, which when released, can flow out of holes 14. The biological solution can be in the form of a gel, a liquid, a powder, a cream, a paste, a slurry, or any other type of solution which can be dispersed through holes 14.

FIG. 2C shows a side cross-sectional view of the first beam 10 without the plunger inserted therein. Optionally there is a container 27 for the biological solution. In this embodiment, container 27 has at least two flanges at the end 27.4 and 27.5 which keep the biological solution from flowing out the back of the container 27. Thus, when the plunger 20 is inserted therein, it punctures container 27 which sends the biological solution out the sides and through holes 14. Alternatively, instead of a container 27, there can be a film 15.1 disposed inside of the housing. The film 15.1 keeps the biological solution from flowing down towards end 17 when the plunger is inserted therein. Instead, the biological solution flows out of holes 14 and into the adjacent affected area. This view also shows threads 31 which sit inside of body section 13. Threads 31 are configured to mesh with threads 21 when the plunger 20 is inserted into the body section 13 and then screwed into the body section 13 so that threads 31 mesh with threads 21.

FIG. 2D shows an end view of container 27 which has an inner sheath 27.1 and an outer sheath 27.3. The outer sheath 27.3 and the inner sheath 27.1 have the biological solution sandwiched in between. When the inner sheath is compressed by the plunger, it causes the biological solution to flow outside of the outer sheath and through holes 14.

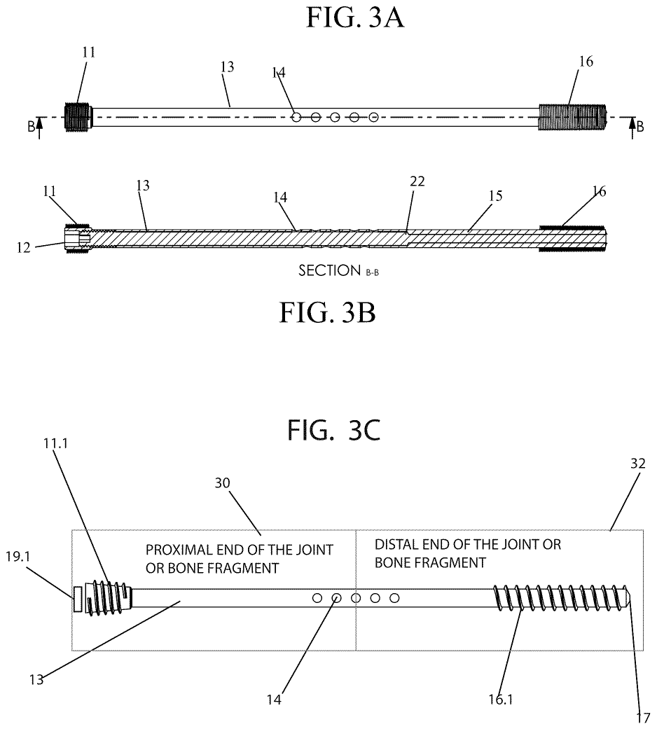

FIG. 3A is a side view of the embodiment of FIG. 1 which has the two parts fully assembled. Holes 14 are shown positioned along body section 13 as well. There are also shown threads 16 as well. Threads 16 are variable pitched relative to threads 11 in order to improve compression with the device.

FIG. 3B is a side cross-sectional view of the embodiment of FIG. 1 which has the two parts fully assembled showing body section 13. Holes 14 are positioned along the body section 13. There is shown surface 22 which is positioned adjacent to stopper 15 such that stopper 15 stops the movement of surface 22 of plunger 20. There is also shown threads 16 on body section 13. Ultimately, while this implant can be used in many different body parts, in at least one embodiment, the implant 10 can be used such that the threaded section or threads 11 are coupled to the proximal bone to be joined. In addition, at the opposite end the threaded section 16 is inserted into a distal bone to be joined by that same patient. This view also shows wrench interface 12 is on part plunger 20. Wrench interface 12 allows for it to receive a screwdriver such as a hex driver to be inserted therein to further adjust and turn the plunger section 20 thereby causing the entire construct to become stronger under loading conditions.

FIG. 3C shows a side view of another embodiment, which shows the proximal end having threads 11.1 which are of a different pitch and frequency from threads 16.1. This view also shows an optional end cap 19.1 which can be fit into wrench interface 12 to close wrench interface 12 off.

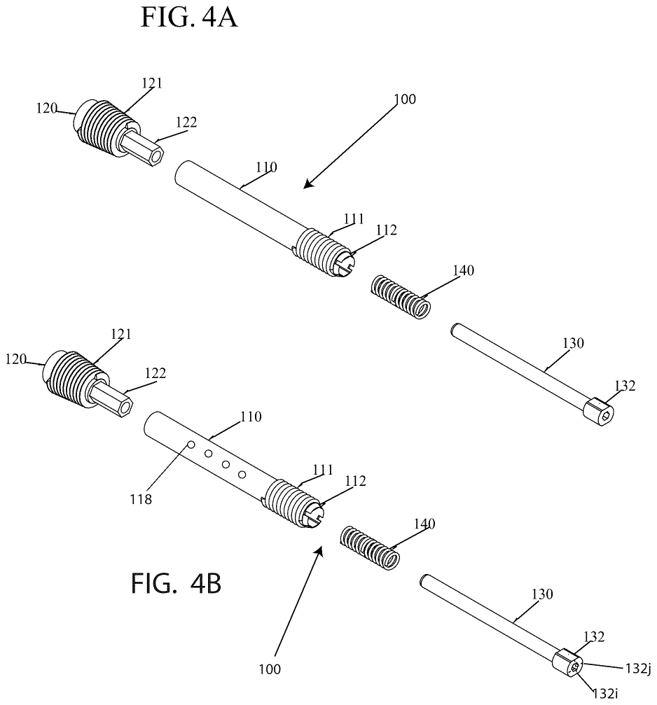

FIG. 4A is a side perspective view of another embodiment of an implant 100 of the invention. This embodiment of the implant 100 includes body section 110 having threads 111 and a cutting tip 112. There is also shown a plunger 130 as well as translation faces 132 positioned on the end of plunger 130. The plunger slides into body 110 and sits on and is biased by spring or other biasing internal element 140 which sits inside of body section 110. There is also shown end cap 120 having threads 121 and anti-rotation surfaces 122.

FIG. 4B shows another exploded view of another embodiment which shows body section 110, holes 118, threads 111, and cutting tip 112. There is also shown spring or other biasing internal element 140 as well as plunger 130 and translation faces 132 positioned on the end of plunger 130. When plunger 130 is inserted into body section 110, it can either drive existing biological solution out of the housing and through holes 118 or puncture a container of biological solution such as that shown in FIG. 2C, causing the biological solution to flow out from a container such as container 27.

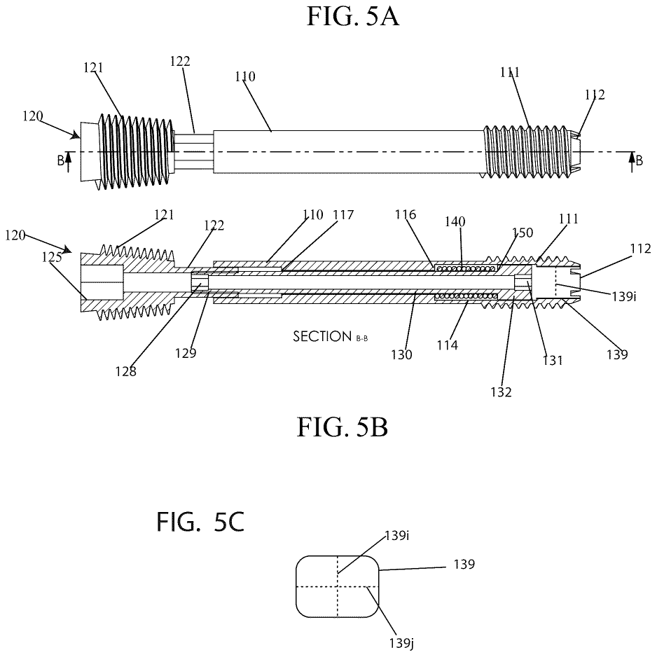

FIG. 5A is a side view of the parts of FIG. 4, showing the device in its expanded state. This view shows body section 110 which has cap 120 inserted therein. Cap 120 has threads 121, as well as anti-rotation feature 122. Body section 110 includes threads 111 as well as cutting tip 112.

FIG. 5B is a side cross-sectional view taken along line B-B of FIG. 5A, showing the device in its expanded state with corresponding spring or other biasing internal element 140 in its compressed state. In this view, there is shown body section 110, which has an end cap cut out 117, which is configured to receive anti-rotation feature 122 on end cap 120. End cap 120 includes wrench interface 125 which is configured to receive a screwdriver head such as a hex or star configuration. End cap 120 has internal threads 129 configured to receive corresponding external threads on plunger 130. Body section 110 has plunger cut out 114 which is configured to receive spring or other biasing internal element 140 while still allowing plunger 130 to fit inside. The plunger 130 and its translation faces 132 would fit inside the body section 110. On the inside of the plunger 130 is a wrench interface 131, which is configured to receive a screwdriver head such as a hex or star configuration. At an end of translation faces 132 is a stopper 150 which is configured to brace against spring or other biasing element 140. Opposite stopper 150 is opposite end face 116 which is configured to brace opposite spring or other biasing element 140. Opposite wrench interface 131 is a wrench interface 128, which is configured to receive a screwdriver head such as a hex or star configuration.

FIG. 5C is a cross-sectional view of the profile of the internal face 139 of the body section 110. This view shows that the internal surface or face 139 of the body section 110 has a profile that is not symmetrical. Instead, the expanse in a first direction 139i of the internal surface 139 is smaller than the expanse 139j of in a direction substantially transverse to the direction of the expanse 139i. The differences in these expanses allows for the accommodation of the oblong translation interface 132 which has a first dimension 132i which is smaller than the second, substantially transverse direction 132j. Thus, when the plunger 130 is turned, the translation faces 132 turn and then are released inside of the body section so that the plunger 130 is allowed to slide inside of the body section 110 as driven by spring 140.

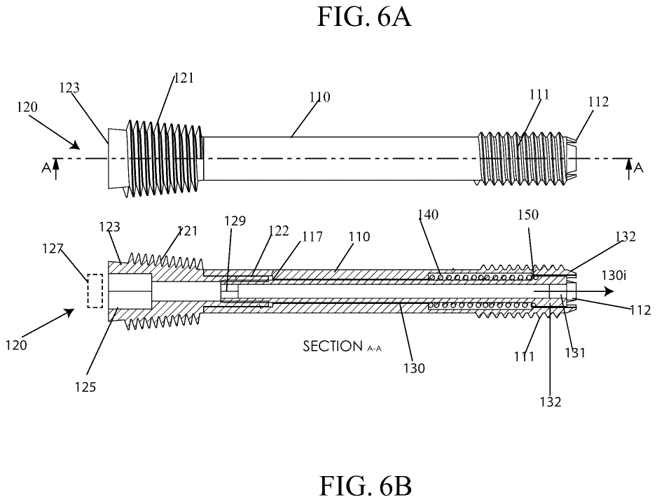

FIG. 6A is a side view of the parts of FIG. 4, showing the device in its compressed state. This view shows body section 110 which shows an optional end cap 120. Cap 120 has threads 121, as well as anti-rotation feature 122. Body section 110 includes threads 111 as well as cutting tip 112. FIG. 6B is a side cross-sectional view taken along line A-A of FIG. 6A, showing the device in its compressed state with the spring or other biasing internal element 140 in its expanded state. All elements shown in FIG. 6B are identical to the corresponding elements in FIG. 5B. As the spring or other internal biasing element 140 expands, the plunger 130's translation faces 132 slide along the body 110's translation faces 133, and the plunger 130 and end cap 120 are biased in the direction of arrow 130i.

When body 110 is positioned adjacent to a bone and screwed into an adjacent bone via cutting tip 112 and threads 111, it causes this body section 110 to be secured at one end to an adjacent bone. The opposite end of this implant is then secured to an adjacent bone via threads 121 on end cap 120. While this implant 100 can be used in many different parts of a person's body in at least one embodiment, the threads 121 can be inserted into the metatarsal bone of a patient. At an opposite end, threads 111 can be inserted into an opposite talus bone. When the spring 140 is released, it compresses both the body section 110 and the end cap 120 together, thereby drawing the metatarsal bone and the talus bone together. There is also shown an optional closing cap 127, which is configured to be fit into the wrench interface 125 on the end cap 120, thereby closing the free end of body 110.

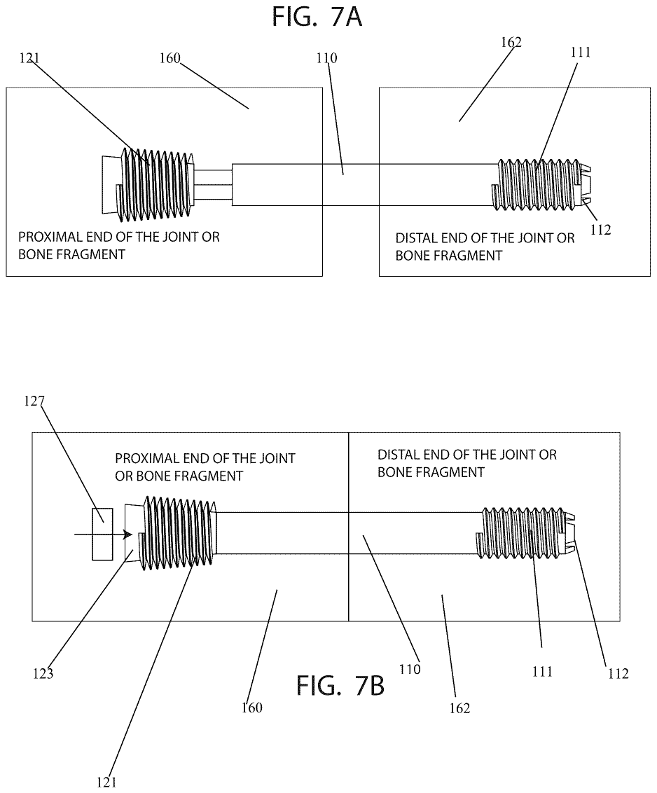

FIG. 7A shows a side view of the embodiment of FIG. 4A in the expanded state as shown in FIG. 5A. In this view there is shown end cap 120, having an end section 123. End cap 120 has outer threads 121. There is also a body section 110 and an additional set of threads 111 at the opposite or distal end. There is also shown a cutting tip 112 at the end adjacent to threads 111.

FIG. 7B shows a side cross-sectional view of the embodiment shown in FIG. 4A in the contracted state as shown in FIG. 6A. This view shows body section 110 having threads 121 and 111. There is also shown an end cap 127 which can be inserted into back end 123 into the wrench interface 125 to cap this wrench interface (See FIG. 6B). The differences between FIGS. 7A and 7B shows that the contraction of the implant draws the proximal end 160 towards the distal end 162. In at least one embodiment the proximal end can be in the form of a metatarsal bone while the distal end is coupled to a bone adjacent to the proximal end or a talus bone. While this implant can be used to draw two bones together such as a metatarsal bone and an adjacent talus bone, the implant can be inserted into other body areas to draw other types of bones together as well.

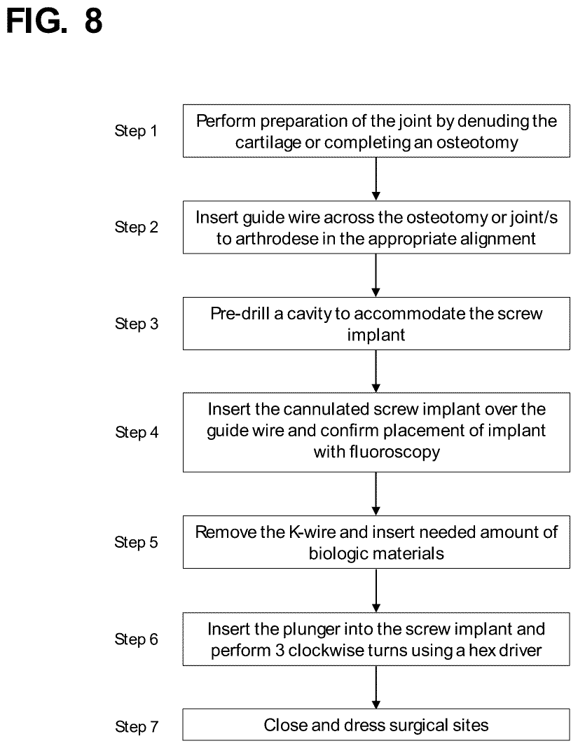

FIG. 8 is a view of the process for inserting the embodiment of FIGS. 1A-3C, excluding FIG. 1D. In the first step the surgeon can prepare the joint by denuding the cartilage or completing an osteotomy. Next, in step 2 the surgeon inserts a guidewire across the osteotomy or joint/s to arthrodese in the appropriate alignment. Next, in step 3 the surgeon can pre-drill a cavity to accommodate the screw implant. Next, in step 4 the surgeon drills the cannulated screw implant such as implant 10 over the guide wire and confirms the placement against fluoroscopy. Next, in step 5 the surgeon can remove the guidewire and insert the needed amount of biologic materials. Next, in step 6 the surgeon can insert the plunger into the screw implant and perform three clockwise turns using a hex driver. Next, in step 7 the surgeon can close and dress the surgical sites.

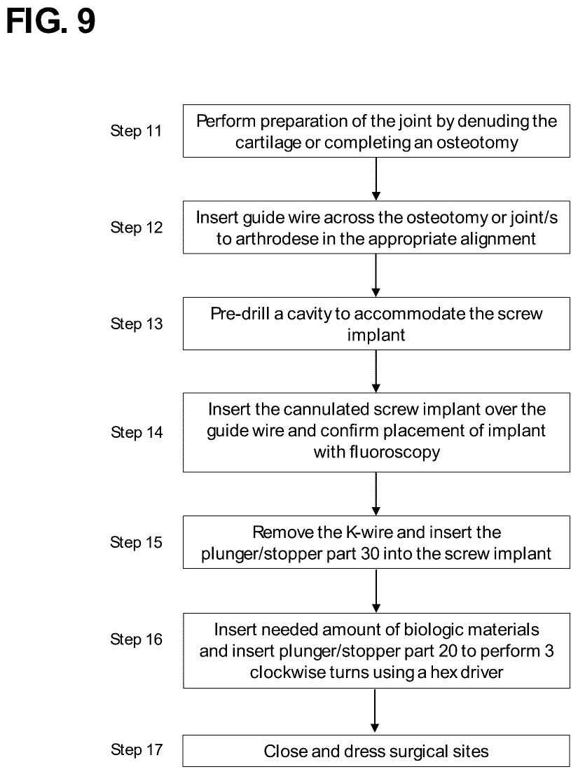

FIG. 9 is a view of the process for inserting the embodiment of FIG. 1D. In step 11 the surgeon can perform preparation of the joint by denuding the cartilage or completing an osteotomy as needed. Next, in step 12 the surgeon inserts a guidewire across the osteotomy or joint/s to arthrodese in the appropriate alignment. Next, in step 13 the surgeon can pre-drill a cavity to accommodate the screw implant. Next, in step 14 the surgeon drills the cannulated screw implant such as implant 10 over the guide wire and confirms the placement against fluoroscopy. Next, in step 15 the surgeon can remove the guidewire and insert the plunger/stopper part 30 into the implant 10. Next, in step 16 the surgeon can insert the needed amount of biologic materials and insert the plunger/stopper part 20 to perform 3 clockwise turns using a hex driver. Next, in step 17 the surgeon can close and dress the surgical sites.

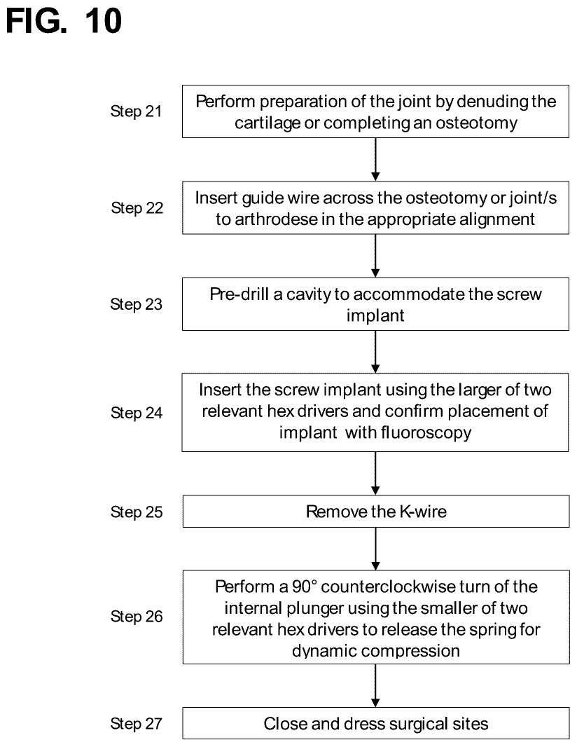

FIG. 10 is a view of a surgical procedure for implanting the embodiment of FIGS. 4A-6B. For example, in step 21 the surgeon can perform preparation of the joint by denuding the cartilage or completing an osteotomy as needed. Next in step 22 the surgeon can insert the guidewire across the osteotomy or joint/s to arthrodese in the appropriate alignment. Next, in step 23 the surgeon can pre-drill the cavity to accommodate the screw implant. Next, in step 24 the surgeon can insert the implant 100 using the larger of the two hex drivers along the end cap 120. Next, in step 25 the surgeon can remove the guide wire. Next, in step 26 the surgeon can perform a 90-degree counterclockwise turn of the internal plunger using the smaller of the two relevant hex drivers to release the spring for dynamic compression. Next, in step 27 the surgeon can close and dress the surgical sites.

Accordingly, while at least one embodiment of the present invention have been shown and described, it is to be understood that many changes and modifications may be made thereunto without departing from the spirit and scope of the invention as defined in the appended claims.

What is claimed is:

1. An implant comprising:
a body section having a hollow internal region;
a plunger configured to be inserted inside of said body section, said plunger being substantially cylindrical and having a first section having a first diameter and a second section having a second diameter wherein said first diameter is smaller than said second diameter;
wherein when said plunger is inserted into said body section it inserts into said internal hollow region of said body section to serve as a strengthening rod for said body section.

2. The implant as in claim 1, wherein said body section has a longitudinal axis, and wherein said body section further comprises a plurality of holes extending along said longitudinal axis of said body section.

3. The implant as in claim 2, wherein said plurality of holes on said body section are positioned in at least two radial positions along said body section and in at least to axial positions on said body section.

4. The implant as in claim 2, wherein said body section is configured to receive at least one biological solution inside of the body section, wherein when said plunger is inserted into said body section said plunger drives said biological solution outside of said plurality of holes on said body section.

5. The implant as in claim 1, wherein said internal hollow region on said body section comprises a first section having a first diameter and a second section having a second diameter, wherein said first section has a diameter that is smaller than said second section.

6. The implant as in claim 4, wherein said diameter of said first section is configured to be smaller than a diameter of said plurality of holes.

7. The implant as in claim 6, further comprising at least one container, wherein said at least one container is configured to store said biological solution inside of said body section wherein said container of biological solution is configured to release said biological solution when it is impacted by said plunger.

8. The implant as in claim 6, further comprising at least one foil, said at least one foil covering an opening of said first section of said body section, said foil for holding said biological solution against flowing into said first section of said body section.

9. The implant as in claim 1, wherein said plunger has a plurality of threads and said body section has a plurality of threads on an inside face of said body section so that said threads on said plunger mesh with said threads on said inside face of said body section so as to lock said plunger inside of said body section.

10. The implant as in claim 1, further comprising a cap, said cap for insertion into said body section after said plunger is inserted and screwed into said body section.

11. An implant comprising:
a body section having a hollow internal region, said body section having an outer housing, a cutting tip;
a plunger configured to be inserted inside of said body section;
at least one biasing element configured to be fit inside of said hollow internal region of said body section;
wherein when said plunger is inserted into said body section, said plunger compresses said biasing element inside of said body section, and wherein said biasing element biases said plunger and said body section in a first position.

12. The implant as in claim 11, wherein said biasing element is a spring.

13. The implant as in claim 12, wherein said body section further comprises a plurality of threads.

14. The implant as in claim 13, further comprising an end cap.

15. The implant as in claim 14, wherein said end cap further comprises threads.

16. The implant as in claim 15, wherein said end cap further comprises a section having anti-rotation surfaces.

17. The implant as in claim 16, wherein said end cap further comprises a wrench interface.

18. The implant as in claim 17, wherein said plunger further comprises translation faces and wherein said body section has internal faces which accommodate said translation faces of said plunger, such that when said plunger rotates, it releases said plunger inside of said body section so that said plunger can be driven axially inside of said body section by said spring.

19. The implant as in claim 14, further comprising a cap, wherein said cap is configured to be inserted into a wrench interface of said end cap.
